(19) United States
(12) Patent Application Publication
(10) Pub. No.: US 2024/0215665 A1
(43) Pub. Date: Jul. 4, 2024

(54) SYSTEM AND METHOD FOR MANAGING OPERATING CONDITION PARAMETERS IN MEDICAL DEVICES

(71) Applicants: Mark T. McBride, Coronado, CA (US); Glenn Butler, Hollitson, MA (US); John Roughneen, Dennis, MA (US)

(72) Inventors: Mark T. McBride, Coronado, CA (US); Glenn Butler, Hollitson, MA (US); John Roughneen, Dennis, MA (US)

(21) Appl. No.: 18/392,270

(22) Filed: Dec. 21, 2023

Related U.S. Application Data
(60) Provisional application No. 63/436,776, filed on Jan. 3, 2023.

(51) Int. Cl. A41D 13/12; A41D 13/002; A41D 13/015; A41D 13/11; A61B 17/00

(52) U.S. Cl. CPC A41D 13/1218; A41D 13/0025; A41D 13/0158; A41D 13/1153; A61B 17/00; A41D 2200/20; A61B 2560/0214; A61B 2562/166

(57) ABSTRACT

A helmetless support and ventilation system for use with surgical hoods and gowns, including a surgical gown, a surgical hood operatively connected to the surgical gown, wherein the hood is located over a head and neck area of a wearer such that the head and neck area of the wearer are substantially enclosed within the hood, a ventilation system located within the surgical gown and the surgical hood for providing ventilation air within the surgical gown and the surgical hood, wherein the ventilation system is retained by shoulders of the wearer of the ventilation system in order to provide ventilation air within the surgical gown and surgical hood, and an operating parameter measurement assembly operatively connected to the ventilation system, wherein the operating parameter measurement assembly is located within the surgical hood.

### CROSS-REFERENCE TO RELATED APPLICATION

This application claims benefit of U.S. Patent Application 63/436,776, filed on Jan. 3, 2023, the disclosure of which is hereby incorporated by reference in its entirety to provide continuity of disclosure to the extent such a disclosure is not inconsistent with the disclosure herein.

### FIELD OF THE INVENTION

The present invention is generally related to a system and method for managing operating condition parameters in medical devices. The system would keep track of operating condition parameters (or air quality) within a medical device such as a surgical hood, wherein the operating condition parameters include, but are not limited to, carbon dioxide (CO₂), temperature, humidity, oxygen (O₂), volatile organic compounds (VOCs), and/or air pressure. The system would provide feedback to the user regarding the operating condition parameters of the system while the system is being used so that the user is aware of the carbon dioxide (CO₂), temperature, humidity, oxygen (O₂), VOCs, and/or air pressure conditions within the medical device especially if the user is wearing the medical device.

For example, if the medical device is a surgical hood having a ventilation system and the user is wearing the surgical hood, it would be desirable for the user to be made aware of the operating parameter conditions within the surgical hood (e.g., carbon dioxide (CO₂), temperature, humidity, oxygen (O₂), VOCs, and/or air pressure) while the ventilation system is being operated. In this manner, if one of the operating parameter conditions within the hood exceeds a pre-determined threshold, the medical device could then interact with the ventilation system in order to attempt to correct the operating parameter condition that has exceeded its pre-determined threshold within the hood. Furthermore, the system could be equipped with an alarm (or alarms) such as an audible and haptic (vibrate) alarm that will alert the user that an operating parameter condition has exceeded a pre-determined threshold and/or that the system has attempted to correct the operating parameter condition that has exceeded its pre-determined threshold, but the system has not been successful in correcting the operating parameter condition that has exceeded its pre-determined threshold. In this latter instance, the user may need to remove the medical device and have maintenance performed on the medical device. Finally, the system would be able to collect information about the operating parameter conditions of the medical devices for user edification and/or product improvement.

### BACKGROUND OF THE INVENTION

Prior to the present invention, as set forth in general terms above and more specifically below, it is known to employ various types of hoods and one-piece gowns or togas for use during medical procedures. The gowns are designed to cover the wearer completely and sterilely when they are attached to the hood. Currently, a helmet or other similar head support structure is donned by the wearer and the one-piece gown and the hood are conventionally attached to the helmet or other similar head support structure. Furthermore, it is known to provide a ventilation system that is also attached to the helmet or other similar head support structure or attached to the wearer.

When the hood and gown are donned by the wearer, it is important that the hood and gown completely and sterilely cover the wearer, as discussed above. In this manner, a closed area is created around the wearer's head and neck areas. Ventilation must be provided within this closed area so that the wearer can wear the hood and gown and still be able to properly perform the medical procedure without having to worry about encountering high levels of carbon dioxide (CO₂), temperature, humidity, oxygen (O₂) VOCs. and/or air pressure within the closed area.

Furthermore, it is known that medical devices need to be properly installed and maintained to function properly. A medical device that is not properly installed or maintained may fail at a very inopportune time, which could lead to a serious incident during a medical operation or procedure. For example, if the medical device is not properly installed or maintained, the wearer may experience high levels of carbon dioxide (CO₂), temperature, humidity, oxygen (O₂), VOCs, and/or air pressure within the closed area.

It is a purpose of this invention to fulfill these and other needs in the medical device art in a manner more apparent to the skilled artisan once given the following disclosure.

The preferred system and method for managing operating condition parameters in medical devices, according to various embodiments of the present invention, offers the following advantages: ease of use; the ability to keep track of operating condition parameters in medical devices; the ability to automatically correct the operating condition parameter in the medical device without user intervention, if needed; the ability to provide feedback regarding the operating condition parameters in the medical device for wearer edification, preventative maintenance, and or product improvement: and the ability to provide audible and haptic (vibrate) alerts. In fact, in many of the preferred embodiments, these advantages are optimized to an extent that is considerably higher than heretofore achieved in prior, known systems and methods for managing operating condition parameters in medical devices.

### BRIEF DESCRIPTION OF THE DRAWINGS

The above-mentioned features and steps of the invention and the manner of attaining them will become apparent, and the invention itself will be best understood by reference to the following description of the embodiments of the invention in conjunction with the accompanying drawings, wherein like characters represent like parts throughout the several views and in which:

FIG. 1 is a schematic, isometric view of a helmetless support for use with surgical hoods and gowns, according to one embodiment of the present invention;

FIG. 2 is a schematic, isometric view of a ventilation system for use with surgical hoods and gowns, constructed according to an embodiment of the present invention;

FIG. 3 is a schematic, side view of the ventilation system for use with surgical hoods and gowns illustrating the adjustable face vent air flow levers, constructed according to an embodiment of the present invention;

FIG. 4 is a cut-away view, taken along lines 4-4 of FIG. 3 of the ventilation system for use with surgical hoods and gowns illustrating the adjustable face vent air flow levers, constructed according to an embodiment of the present invention:

FIG. 5 is a schematic, isometric, back view of the ventilation system for use with surgical hoods and gowns with the back cover removed illustrating the power module, constructed according to an embodiment of the present invention;

FIG. 6 is an isometric, back view of the ventilation system for use with surgical hoods and gowns with the back cover removed illustrating the printed circuit board (PCB) module, constructed according to an embodiment of the present invention;

FIG. 7 is a schematic illustration of the ventilation system for use with surgical hoods and gowns illustrating the operating condition parameter measurement device being located adjacent to the wearer in one embodiment, constructed according to an embodiment of the present invention;

FIG. 8 illustrates one embodiment for a method for managing operating parameters in medical devices; and

FIG. 9 illustrates an embodiment of a computing system configured with the example systems and/or methods disclosed.

### DETAILED DESCRIPTION OF THE PREFERRED

### Embodiments of the Invention

In order to address the shortcomings of the prior, known systems and methods for managing operating parameters in a medical device, it would be desirable to utilize a system and method for managing operating condition parameters in medical devices. The system would keep track of operating condition parameters (or air quality) within the surgical hood, wherein the operating condition parameters include, but are not limited to, carbon dioxide (CO₂), temperature, humidity, oxygen (O₂), volatile organic compounds (VOCs), and/or air pressure. The system would provide feedback to the user regarding the operating condition parameters of the system while the system is being used so that the user is aware of the carbon dioxide (CO₂), temperature, humidity, oxygen (O₂), VOCs, and/or air pressure conditions within the medical device especially if the user is wearing the medical device.

For example, if the medical device is a surgical hood having a ventilation system and the user is wearing the surgical hood, it would be desirable for the user to be made aware of the operating parameter conditions within the surgical hood (e.g., carbon dioxide (CO₂), temperature, humidity, oxygen (O₂), VOCs. and/or air pressure) while the ventilation system is being operated. In this manner, if one of the operating parameter conditions within the hood exceeds a pre-determined threshold, the medical device could then interact with the ventilation system to attempt to correct the operating parameter condition that has exceeded its pre-determined threshold within the hood. Furthermore, the system could be equipped with an alarm (or alarms) such as an audible and haptic (vibrate) alarm that will alert the user that an operating parameter condition has exceeded a pre-determined threshold and/or that the system has attempted to correct the operating parameter condition that has exceeded its pre-determined threshold, but the system has not been successful in correcting the operating parameter condition that has exceeded its pre-determined threshold. In this latter instance, the user may need to remove the medical device and have maintenance performed on the medical device. Finally, the system would be able to collect information about the operating parameter conditions of the medical devices for user edification and/or product improvement.

A unique aspect of the present invention is that the pre-determined threshold can be determined according to, but not limited to, the following:
1. A value set by the user for comfort;
2. A value set by the hospital/system administrator policy;
3. A value based upon best practices; and/or
4. A value based upon compliance with Occupational Safety and Health Administration (OSHA) policies.

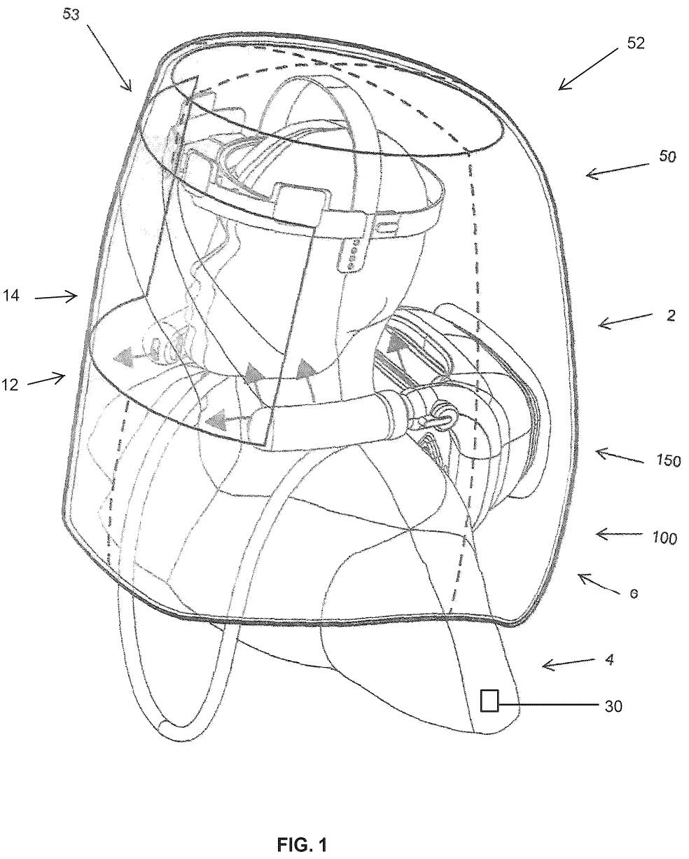

Referring now to FIG. 1-7, there is illustrated a helmetless support system 2 for use with surgical hoods and gowns. The helmetless support system 2 for use with surgical hoods and gowns can be used to support the one-piece surgical gown 4 and the surgical hood 14 without the need for the wearer 6 to wear a helmet. In this manner, one-piece surgical gown 4 and the surgical hood 14 completely and sterilely covers the head, neck, and torso of the wearer 6 when donned by the wearer 6. Also, the one-piece surgical gown 4 and the surgical hood 14 includes a clear faceplate 12. The helmetless support 2 further includes a helmetless surgical hood and gown support having a flexible headband 52 with attached lightweight front offsets 53 in front that can be releasably attached to the faceplate 12. Furthermore, the front offsets 53 are used to provide air circulation around head of the wearer 6.

### Helmetless Surgical Hood and Gown Support

As shown in FIG. 1, helmetless support 2 for use with surgical hoods and gowns includes, in part, surgical gown 4, wearer 6, gown wireless identification system 30, and helmetless surgical hood and gown support 50. It is to be understood that surgical gown 4 is constructed of any suitable, durable, medical grade material. It is to be further understood that the surgical gown 4 is to be constructed into a one-piece design that will completely and sterilely cover the wearer when attached to the hood 14. Finally, it is to be understood that gown wireless identification system 30 is a conventional wireless identification system such as an RFID tag that can be conventionally attached to the surgical gown 4.

With respect to helmetless surgical hood and gown support 50, helmetless surgical hood and gown support 50 includes, in part, flexible, adjustable band 52 and front offsets 53. Preferably, flexible band 52 is constructed of any suitable, durable, flexible, medical grade material. An important feature of flexible band 52 being that it comfortably fits around the head of the wearer 6, but still is capable of securely holding surgical gown 4 and surgical hood 14 once the surgical gown 4 and surgical hood 14 have been attached to helmetless surgical hood and gown support 50 and then placed over the wearer, as will be discussed in greater detail later. In particular, it is important that flexible band 52 be able to securely hold hood 14 off of the head of wearer 6 and allow the air to flow around the head of wearer 6, as will be discussed in greater detail later.

### Helmetless Surgical Hood and Gown Ventilation System

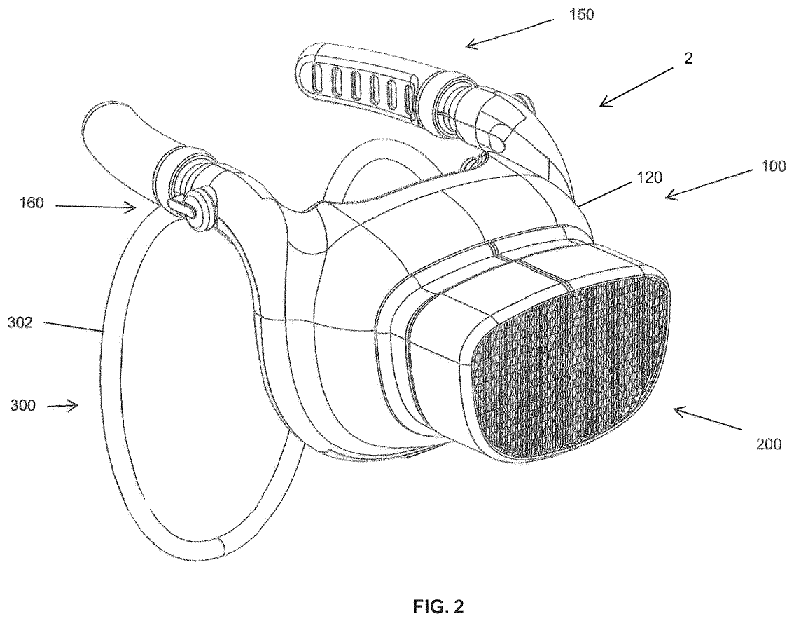

Referring now to FIGS. 2-7, there is illustrated a ventilation system 100 for use with helmetless support system 2. The ventilation system 100 is constructed such that the fan speed can be controlled by the wearer 6 and/or the system 2 once the gown 4 and hood 14 have been donned. A face vent module 150 is used as a “yoke” to support the ventilation system 100 on the shoulders of the wearer 6 (FIG. 1). Finally, the wearer 6 can control the output from each of the various output apertures in face vent module 150 and neck vent module 350 in the ventilation system 100, as will be discussed in greater detail later.

As shown in FIGS. 2-7, helmetless support system 2 for use with surgical hoods and gowns having ventilation system 100 includes, in part, protective casing 120, face vent module 150, air filtration module 200, power module 250, yoke module 300, neck vent module 350, air flow generation module 450, printed circuit board (PCB) module 500, and the operating parameter measurement assembly 600.

A unique aspect of the present invention is the location of the ventilation system 100 with respect to the surgical gown 4 and surgical hood 14. As shown in FIG. 1, the ventilation system 100 is almost completely located inside of the surgical gown 4 and surgical hood 14.

### Protective Casing 120

With respect to protective casing 120, protective casing 120, preferably, is constructed of any suitable, durable, high strength, shock resistant, UV resistant, medical grade polymeric material. It is to be understood that protective casing 120 is used to encase ventilation system 100 to provide protection for air filtration module 200, power module 250, neck vent module 350, air flow generation module 450, and printed circuit board (PCB) module 500.

### Face Vent Module 150

Regarding face vent module 150, as shown in FIGS. 2-6, face vent module 150, includes, in part, removable face vents 152, face vent openings 154, face vent connectors 156, face vent adaptors 158, face vent air flow adjustors 160, and face vent air flow adjuster lever 162. Preferably, face vents 152 and face vent connectors 156 are constructed as a single-piece construction and are constructed of any suitable, durable, lightweight, medical grade, and washable material. Also, face vent openings 154 are formed in removable face vents 152 by conventional techniques such as forming, stamping, molding, or the like. Face vent adaptors 158, preferably, are constructed of any suitable, durable, high strength, medical grade material and are permanently connected to protective casing 120 near face vent air flow adjustors 160 and face vent air flow adjuster levers 162. Finally, face vent air flow adjustors 160 and face vent air flow adjuster lever 162, preferably, are constructed of any suitable, durable, high strength, medical grade material.

A unique aspect of the present invention is the use of removable face vents 152. In particular, removable face vents 152 are constructed in such a manner that allows the removable face vents 152 to be easily removed from the face vent adaptors 158 so that the removable face vents 152 can be cleaned, disinfected, and sanitized prior to the next usage of the helmetless support 2 for use with surgical hoods and gowns having ventilation system 100. Once the removable face vents 152 have been cleaned, disinfected, and sanitized, the removable face vents 152 can be easily slid onto the face vent adaptors 158 by locating the face vent connectors 156 on the face vent adaptors 158.

A further unique aspect of the present invention is the use of face vent air flow adjustors 160 and face vent air flow adjuster lever 162. In particular, the wearer 6 can adjust the amount of air flow that is being emitted out of the removable face vents 152 through the use of vent air flow adjustor 160 and face vent air flow adjuster lever 162. In this manner, the wearer 6 can conventionally manipulate face vent air flow adjuster lever 162 so that the amount of air flow is adjusted. For example, the wearer 6 may push/pull the face vent air flow adjuster lever 162 upwards which will cause the amount of air flow being emitted out of the removable face vents 152 to be reduced. Conversely, the wearer 6 may push/pull the face vent air flow adjuster lever 162 downwards which will cause the amount of air flow being emitted out of the removable face vents 152 to be increased.

### Air Filtration Module 200

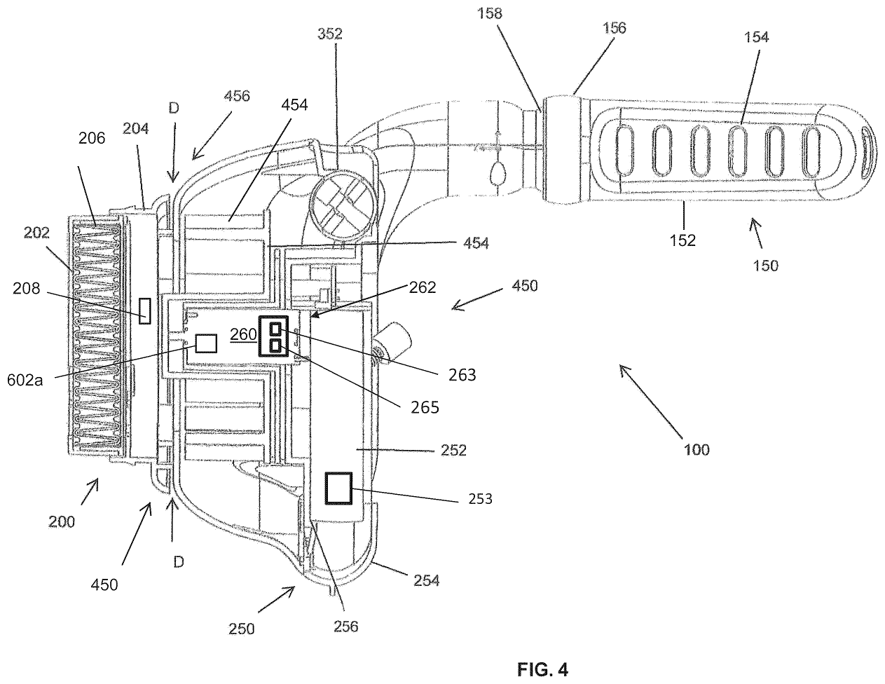

With respect to air filtration module 200, as shown in FIG. 4, air filtration module 200, includes, in part, air filter 202, air filtration adaptor 204, filter casing 206, and air filtration module wireless identification system 208. Preferably, air filter 202 is a HEPA (or ULPA) air filter that is located within filter casing 206. Preferably, filter casing 206 is constructed of any suitable, durable, high strength, medical grade material. Preferably, air filtration adaptor 204 is conventionally formed on protective casing 120. Finally, it is to be understood that air filtration module wireless identification system 208 is a conventional wireless identification system such as an RFID tag that can be conventionally attached to or electrically connected to the air filter 202, as will be discussed in greater detail later.

A unique aspect of the present invention is the use of air filtration module 200. In particular, air filtration module 200 can be used to filter out air borne contaminants so that they do enter into the surgical hood 14 and surgical gown 4. As discussed above, only air filter 202 extends outside of the surgical hood 14 (FIG. 1). In this manner, only air going through the air filtration module 200 will be allowed to enter into the surgical hood 14 and surgical gown 4. Also, the air filter 202 can be easily removed and replaced. For example, wearer 6 can simply remove the air filter 202 and the filter casing 206 from the air filtration adaptor 204. The wearer 6 can then replace the used air filter 202 and filter casing 206 with a new air filter 202 and filter casing 206 by simply placing the new air filter 202 and filter casing 206 onto the air filtration adaptor 204. It is to be understood that the air filter 202 and filter casing 206 can be retained on the air filtration adaptor 204 by a snap fit, a threaded connection, a bayonet connection, a slidable connection or the like, as will be discussed in greater detail later.

### Power Module 250

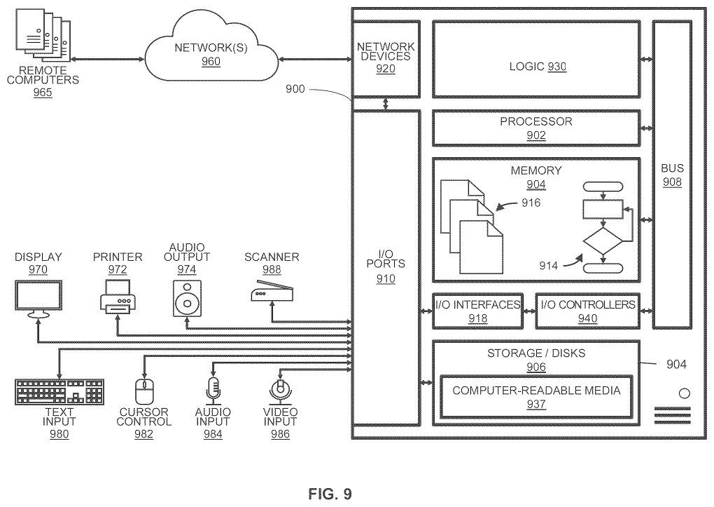

Regarding power module 250, as shown in FIGS. 4-6, power module 250, includes, in part, battery 252, battery sensor 253, battery doors 254, battery lock 256, wireless power module identification system 258, motor sensor/wireless identification system 262, tachometer 263, and a printed control board 265. Preferably, battery 252 is a conventional, rechargeable battery such as a lithium-ion battery or the like that is capable of providing sufficient power to air flow generation module 450, printed circuit board (PCB) module 500, and operating condition parameter measurement assembly 600 for an extended period of time such as 6-8 hours. Battery sensor 253 is a conventional sensor that can be used to monitor the operation of battery 252 to ensure that battery 252 is operating properly. Also, battery doors 254, preferably are constructed of any suitable, durable, high strength, medical grade material. It is to be understood that wireless power module identification system 258 is a conventional wireless identification system such as an RFID tag that can be conventionally attached to or electrically connected to the battery 252, as will be discussed in greater detail later. It is to be understood that instead of an RFID tag, the battery 252 may include a serial number that can be conventionally read/detected using a hardware data line that is configured to be used with the battery 252 through the wireless power module identification system 258. It is to be further understood that motor sensor/wireless identification system 262 includes a conventional printed circuit board 265, a tachometer 263, a RFID tag (not shown) that can be conventionally attached to or electronically connected to the fan motor 260, as will be discussed in greater detail later. Furthermore, printed circuit board 265 is configured with an algorithm that is used to determine the operating speed of the fan motor 260 based on one or more of the previously discussed pre-determined thresholds. Finally, in one embodiment, the algorithm could be updated with information uploaded onto the printed circuit board 265 through an interaction with processor 902 (FIG. 9) either manually or automatically based upon the desired interval in which such information is to be uploaded onto the printed circuit board 265.

A unique aspect of the present invention is the use of battery sensor 253. In particular, battery sensor 253 can be used to monitor the cycles and voltages of battery 250 so that it can be determined when the battery 250 needs to be replaced. Also, the battery sensor 253 can be used in conjunction with the processor 902 (FIG. 9) and printed circuit board (PCB) module 500 to utilize the information from battery sensor 253 in order to alert the user that the battery 250 is not functioning properly or to simply place an order for a replacement for battery 250.

Another unique aspect of the present invention is the use of battery doors 254. Battery doors 254 are conventionally connected to protective casing 120 so that battery doors 254 can swing (or pivot) open so that battery 252 can be easily installed into power module 250 or removed from power module 250. In particular, the wearer 6 can remove battery 252 from power module 250 by opening battery doors 254 and removing battery 252 from power module 250. The battery 252 can then be placed on a conventional battery charger (not shown). Once battery 252 has been fully charged, the wearer 6 can then remove the battery 252 from the battery charger, open the battery doors 254, and slide the battery 252 into the power module 250 so that the battery 252 is securely retained within the power module 250. The wearer 6 then closes the battery doors 254 so that the battery 252 is not exposed to the elements. It is to be understood that a conventional locking mechanism 256 can be used to lock the battery 252 in place in the power module 250 so that the battery 252 does not inadvertently come loose while the ventilation system 100 is being operated.

### Yoke Module 300

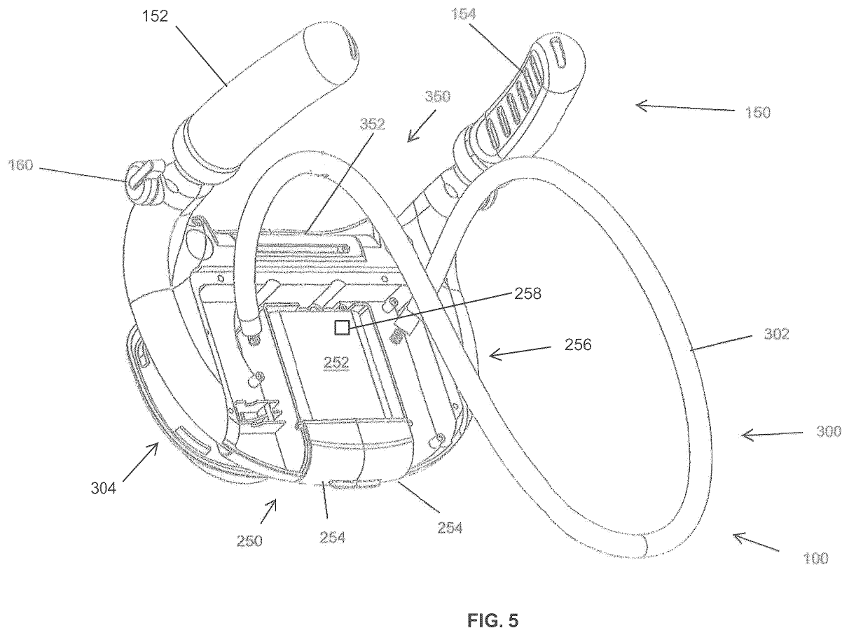

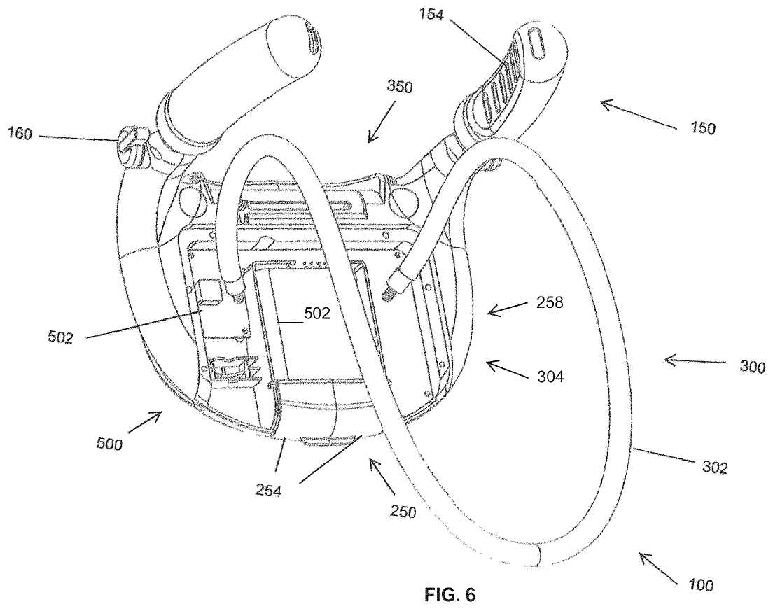

With respect to yoke module 300, as shown in FIGS. 5 and 6, yoke module 300, includes, in part, yoke 302 and yoke connectors 304. Preferably, yoke 302 is constructed of any suitable, durable, high strength, flexible, medical grade material. Preferably, yoke connectors 304 are attached to the back of protective casing 120.

Another unique aspect of the present invention is the use of yoke module 300. In particular, yoke module 300 can be used to assist in retaining ventilation system 100 on the shoulders of the wearer 6. Furthermore, yoke 302 is removably attached to protective casing through the use of yoke connectors 304. In this manner, yoke 302 can be easily attached to and removed from protective casing 120. Furthermore, since yoke 302 is flexible, yoke 302 can be adjusted to fit the upper torso of the wearer 6 so that ventilation system 100 will remain securely retained on the shoulders and the upper torso of the wearer 6. For example, wearer 6 can position the ventilation system 100 with the yoke module 300 installed over his/her head and place the yoke module 300 on the upper torso of the wearer 6 (FIG. 1). The wearer 6 can then pull/push on yoke 302 while yoke 302 is connected to yoke connectors 304 so that yoke 302 firmly contacts the upper torso of the wearer 6 to assist in retaining the ventilation system 100 on the shoulders and upper torso of the wearer 6.

### Neck Vent Module 350

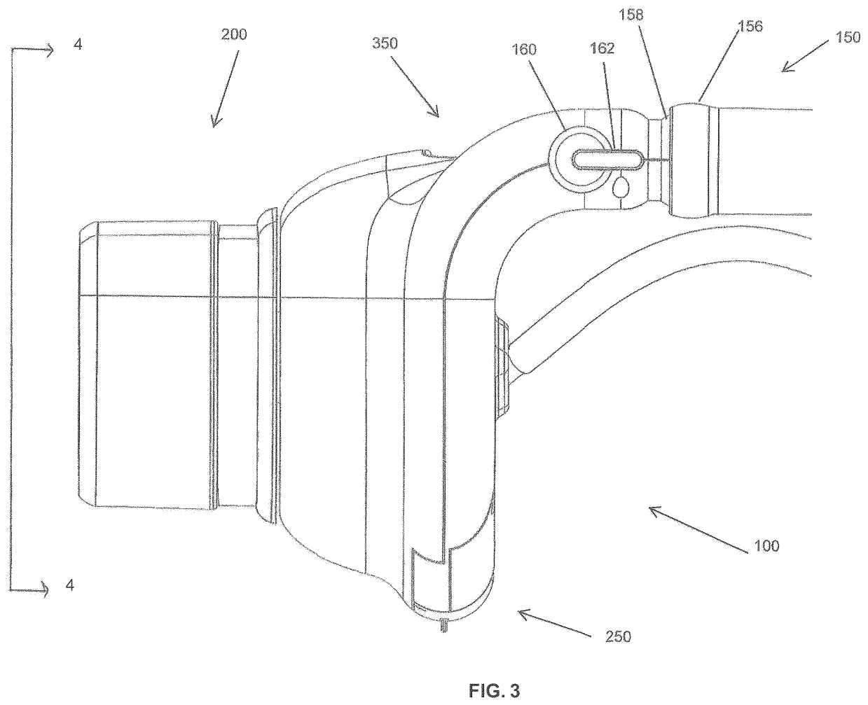

Regarding neck vent module 350, as shown in FIGS. 3-6, neck vent module 350 includes, in part, neck vent 352 (FIG. 4). Preferably, neck vent 352 is constructed of any suitable, durable, high strength, medical grade material.

### Air Flow Generation Module 450

Regarding air flow generation module 450, as shown in FIG. 4, air flow generation module 450 includes, in part, conventional fan motor 260, conventional impeller 454, and back flow opening 456. It is to be understood that battery 252 provides the electrical power to fan motor 260.

Another unique aspect of the present invention is the use of air flow generation module 450. In particular, as the fan motor 260 causes the impeller (or fan) 454 to rotate, the configuration of the impeller 454 causes air to be drawn through the air filter module 200. In this manner, the air filter module 200 can be used to filter the air being drawn into the ventilation system 100. Also, the back flow opening 456 is provided to allow air that is contained within the surgical hood 14 to also be drawn through back flow opening 456 in the direction of arrow D. In this manner, the back flow opening 456 provides for an even greater circulation of the air within the hood 14 while the ventilation system 100 is in operation.

### Printed Circuit Board (PCB) Module 500

With respect to printed circuit board (PCB) module 500, as shown in FIG. 6, printed circuit board module 500, includes, in part, a conventional printed circuit board 502. It is to be understood that printed circuit board 502 can be used to control the ventilation system 100 and interact with gown wireless identification system 30, air filtration module wireless identification system 208, power module wireless identification system 258, motor sensor/wireless identification system 262, and operating condition parameter measurement system 600, as will be described in greater detail later. In particular, printed circuit board 502 can be used to control the speed at which the impeller 454 (FIG. 4) rotates, thereby controlling the velocity of the air being emitted from the face vents 152 and the neck vent 352. It is to be further understood that the printed circuit board 502 is located in the rear of the protective casing 120 so that the printed circuit board 502 can be located adjacent to battery 252. Finally, it is to be understood that the printed circuit board 502 is conventionally retained within the protective casing 102 by conventional fasteners (not shown). Finally, it is to be understood that the printed circuit board (PCB) module 500 can utilize Bluetooth® low energy capabilities in order to allow the printed circuit board (PCB) module 500 to communicate with a mobile application that is conventionally installed on a remote computer 965 (i.e., mobile communication device) (FIG. 9) such as a smartphone, tablet or data collection point.

### Operating Condition Parameter Measurement Assembly 600

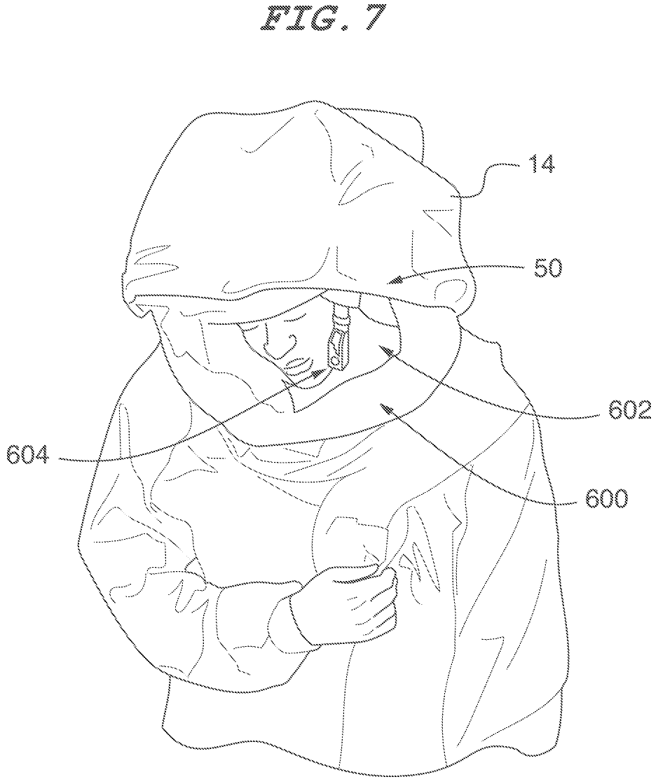

Regarding operating condition parameter measurement assembly 600, as shown in FIGS. 4 and 7, operating condition parameter measurement assembly 600, includes, in part, hood 14, helmetless surgical hood and gown support 50, operating condition parameter sensor 602 (and/or 602a in FIG. 4), and microphone assembly 604. In one embodiment, operating condition parameter sensor 602 and/or 602a can include, but is not limited to, a carbon dioxide (CO₂) sensor, a temperature sensor, a humidity sensor, oxygen (O₂) sensor, volatile organic compounds (VOCs) sensor, and/or an air pressure level sensor. The operating condition parameter sensor 602 (and/or 602a) can be used to measure air quality within hood 14 while the hood 14 is being worn by the user. In one embodiment, the air quality can be related to, but not limited to, a carbon dioxide (CO₂) level, a temperature level, a humidity level, an oxygen (O₂) level, a volatile organic compounds (VOCs) level, and/or an air pressure level within the hood 14 while the hood 14 is being worn by the user.

As shown in FIG. 7, in one embodiment, the operating condition parameter sensor 602 is connected to the helmetless surgical hood and gown support 50 so that operating condition parameter sensor 602 can be securely located adjacent to the mouth of the wearer. It is to be understood that operating condition parameter measurement assembly 600 can be adjusted so as to be able to position the operating parameter condition measurement assembly 600 within a desired distance away from the wearer's mouth. It is to be further understood that while the operating condition parameter sensor 602 is being used in conjunction with the helmetless surgical hood and gown support 50 and hood 14, operating condition parameter sensor 602 can be used on other medical devices located within the hood 14. Furthermore, operating condition parameter sensor 602 could be used in conjunction with filter 202 to measure the quality of the air that is being introduced into filter 202.

In another embodiment, operating condition parameter measurement assembly 600 can also include an operating condition parameter sensor 602a which can also be located adjacent to the fan motor 250 (FIG. 4) instead of being located adjacent to the user's mouth (FIG. 7). In still another embodiment, both operating condition parameter sensor 602 and 602a can be utilized within hood 14. It is to be understood no matter if either or both operating condition parameter sensor 602 and/or 602a are utilized within hood 14, the operating condition parameter sensor 602 and/or 602a should be located within the hood 14 so that the operating condition parameter sensor 602 and/or 602a can monitor the operating condition parameters or air quality (i.e., CO₂ levels, temperature, humidity levels, oxygen (O₂) levels, volatile organic compounds (VOCs), etc.) within the hood 14 while the hood 14 is being used during a medical procedure.

In another embodiment, the operating condition parameter measurement assembly 600 can also be equipped with a microphone assembly 604. In this manner, the microphone assembly 604 can be used to allow the wearer to communicate with other personnel in the area where the medical procedure is being performed and/or personnel who are observing the medical procedure at a location remote from the medical procedure area.

### Operation of System for Managing Medical Device Maintenance and Medical Device Consumables

With respect to the operation of the system for managing medical device maintenance and medical device consumables, attention is directed to FIGS. 1-9. Assume that a medical device such as a surgical gown 4 (FIG. 1) is equipped with a conventional gown wireless identification system 30 having an RFID tag that can be conventionally attached to or electrically connected to the surgical gown 4, as discussed earlier. Secondly, assume that another medical device such as a fan filter 202 (FIG. 4) is equipped with a conventional wireless identification system 208 having an RFID tag that can be conventionally attached to or electronically connected to the air filter 202. Thirdly, assume that a still another medical device such as a battery 252 (FIGS. 4 and 5) is equipped with a conventional power module wireless identification system 258 having an RFID tag that can be conventionally attached to or electronically connected to the battery 252. Fourthly, assume that a fan motor 260 (FIG. 4) is equipped with a motor sensor/wireless identification system 262 having a tachometer 263 and RFID tag that can be conventionally attached to or electronically connected to the fan motor 260. It is to be understood that a processor 902 (FIG. 9) in conjunction with printed circuit board (PCB) module 500 can use the information from tachometer 263 or any other similar device in the motor sensor/wireless identification system 262 to determine the speed at which the motor 260 is operating and compare that speed to the speed at which the motor 260 should be preferably operating in order to determine if the filter 202 is clogged, the motor 206 is damaged, an operating condition parameter threshold has been exceeded, and/or the system 4 is otherwise malfunctioning to alert the user. Finally, assume that gown wireless identification system 30, air filtration module wireless identification system 208, power module wireless identification system 258, motor sensor/wireless identification system 262, and the operating condition parameter measurement assembly 600 are in electrical communication with printed circuit board 502.

In another embodiment, information from tachometer 263 or any other similar device in the motor sensor/wireless identification system 262 can be sent to processor 902 so that processor 902 in conjunction with printed circuit board (PCB) module 500 can control the speed of fan motor 260. In particular, operating condition parameter measurement assembly 600 (i.e., operating condition parameter sensor 602 and/or 602a) can be used to provide information to processor 902 regarding the operating conditions within the hood 14 (FIG. 7). For example, operating condition parameter sensor 602 and/or 602a can be used to detect carbon dioxide (CO₂), temperature, humidity, oxygen (O₂), VOCs, and/or air pressure levels within hood 14. The operating conditions can be monitored while the wearer is donning the hood 14. The operating conditions data from operating conditions parameter sensor 602 and 602a can be transmitted through the printed circuit board (PCB) module 500 to the processor 902. In another embodiment, the processor 902 has been configured with operating condition parameter thresholds (such as CO₂ level should not exceed 1,200 ppm or 2,500 ppm depending upon the desired operating conditions). As discussed above, an algorithm running on the printed control board 265 determines the operating speed of the fan motor 260 based on one or more pre-determined thresholds, and the algorithm could be updated with data from the processor 902 (FIG. 9) and uploaded onto the printed control board 265 at a desired interval.

If at least one of the thresholds of an operating condition (carbon dioxide (CO₂), temperature, humidity, oxygen (O₂), VOCs. and/or air pressure levels) is exceeded, the processor 902 in conjunction with printed circuit board (PCB) module 500 can then interact with the fan motor 260 to adjust the speed of the fan motor 260. In one embodiment, the tachometer 263 can then be utilized to monitor the adjustment in the speed of the fan motor 260 so that the desired adjustment in the speed of the fan motor 260 is achieved. For example, the processor 902 in conjunction with printed circuit board (PCB) module 500 can interact with the fan motor 260 to cause the fan motor 260 to quickly accelerate in speed in order to provide a large amount of fresh air inside of hood 14 if at least one of the thresholds of an operating condition (carbon dioxide (CO₂), temperature, humidity, oxygen (O₂), VOCs, and/or air pressure levels) is exceeded, as discussed earlier. For example, if the CO₂ levels within hood 14 exceed 1,200 ppm (or 2,500 ppm depending upon the operating conditions), the introduction of large amount of fresh air into the hood 14 over a desired period of time may correct the operating condition in which the threshold of this operation condition has been exceeded. The operating parameter sensor 602 and/or 602a will continuously monitor the operating conditions within hood 14 while the wearer is donning the hood 14 to determine that no other operating conditions within hood 14 are exceeding their operating thresholds.

In still another embodiment, the processor 902 in conjunction with printed circuit board (PCB) module 500 can interact with the fan motor 260 to cause the fan motor 260 to gradually accelerate or decelerate in speed in order to provide a desired amount of fresh air inside of hood 14 if at least one of the thresholds of an operating condition (carbon dioxide (CO₂), temperature, humidity, oxygen (O₂), VOCs, and/or air pressure levels) is exceeded. For example, if the CO₂ level within hood 14 exceeds 2,500 ppm, the fan motor 260 can be activated to begin to introduce an amount of fresh air into the hood 14 in order to correct the operating condition in which the threshold of this operation condition has been exceeded. In this example, once it has been determined that the CO₂ levels are below 2,500 ppm, the fan motor 260 can be decelerated or turned off to cause the fan motor 260 to slow or stop the delivery of fresh air into the hood 14. As discussed above, the operating parameter sensor 602 and/or 602a will continuously monitor the operating conditions within hood 14 while the wearer is donning the hood 14 to determine that no other operating conditions within hood 14 are exceeding their operating thresholds.

In another embodiment, the tachometer 263 can then be utilized to monitor a “full” condition. For example, tachometer 263 can be used to detect if filter 202 is clogged, motor 260 is failing, or the like. In any of these instances, the processor 902 in conjunction with printed circuit board (PCB) module 500 can then be configured to send an alert to the user informing the user that a “full” condition exists and needs to be looked at.

In another embodiment, the operating condition information forwarded from the operating parameter sensor 602 and/or 602a to the processor 902 can also be stored in data storage 906 or forwarded to a remote computer 965 associated with the wearer or a system administrator so that the wearer and/or system administrator will have access to the operating conditions within the hood 14 while the wearer is donning the hood 14. For example, the remote computer 965 may be equipped with a graphical user interface (not shown) that will allow the wearer and/or the system administrator to view the operating conditions within the hood 14 in a graphical format or some other similar format that will allow the wearer and/or system administrator to quickly and easily ascertain if there are any operating conditions within the hood 14 that are being exceeded while the wearer is donning the hood. Furthermore, the information regarding the operating conditions can be used for subsequent wearer edification, preventative maintenance, and or product improvement.

In one embodiment, the wearer and/or system administrator can be provided with an operating parameters (or air quality) health score. In this manner, after the medical procedure has been completed, the wearer and/or system administrator can be provided with a color score such as a green/yellow/red score. In this embodiment, the color would provide the wearer and/or system administrator with information about the air quality within hood 14 while the wearer was performing the medical procedure. For example, the color green could be associated with a good health score (the thresholds of an operating condition were not exceeded within the hood 14 during the medical procedure). The color yellow could be associated with a fair health score (only 1 or 2 of the thresholds of an operating condition were exceeded within the hood 14 during the medical procedure). The color red could be associated with a poor health score (many of the thresholds of an operating condition were exceeded within the hood 14 during the medical procedure).

Furthermore, operating conditions could be monitored every 30 seconds (or other desired time period). In this manner, if the wearer was performing a medical procedure that required more than usual manual exertion, it is possible that the wearer's CO₂ output, humidity, oxygen (O₂), VOCs, and/or temperature within the hood 14 might be greater than during a less strenuous medical procedure. The information related to the amount of CO₂ that the wearer is exerting, along with the increased humidity, temperature, oxygen (O₂), and VOC levels, could be useful to the wearer and/or the system administrator so that possibly the speed of the fan motor 260 could be automatically increased in similar medical procedures in the future thereby maintaining the CO₂, humidity, oxygen (O₂), VOC, and/or temperature levels within the hood 14 below the threshold levels.

Another unique aspect of the present invention is that the information obtained for use in determining the operating parameter health score can also be used to create best practices for air quality, threshold, and usage settings. In particular, data that is related to the operating parameters (or air quality) health score data and is temporarily stored in memory 904 under data 916 can be executed by processor 902 from a server (FIG. 9) to determine best practices such as fan speed, optimum CO₂ levels, humidity levels, temperature levels, oxygen (O₂) levels, and/or VOC levels. This data can then be used in conjunction with machine learning techniques and artificial intelligence (AI) algorithms to recommend best practices for consumable operation and management in order to improve air quality within the hood 14 and when using the ventilation system 100.

In still another embodiment, processor 902 can also be configured to send a signal to an alarm (not shown) such as an audible and haptic (vibrate) alarm to provide an alert to the user to inform the user that at least one of the thresholds of an operating condition (carbon dioxide (CO₂), temperature, humidity, oxygen (O₂), VOCs, and/or air pressure levels) has been exceeded. In this instance, if the processor 902 interacts with fan motor 260 upon determining that an operating condition threshold has been exceeded, but the fan motor speed is not adjusted to attempt to correct the operating condition being exceeded, the alarm can provide a backup to the wearer and/or the system administrator that the system 2 needs to be looked at to determine why the system 2 allowed the operating condition to exceed its operating threshold.

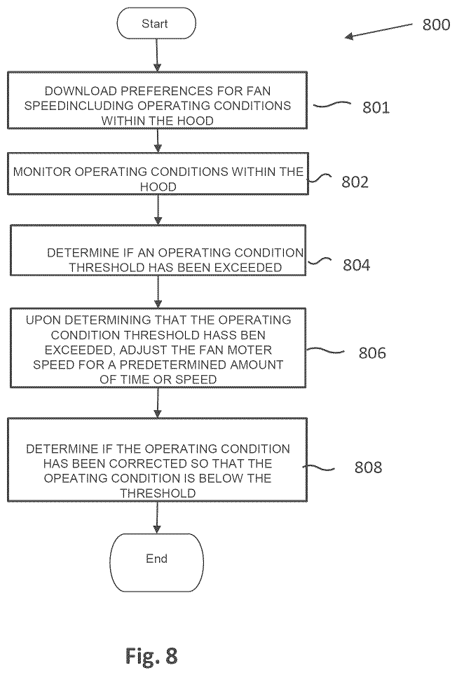

With reference to FIG. 8, a computer-implemented method 800 is illustrated that describes one embodiment for managing operating condition parameters in medical devices. The method 800 is performed by at least a processor of a computer system that accesses and interacts with memories and/or data storage devices. For example, the processor at least accesses and reads/writes data to the memory and processes network communications to perform the actions of FIG. 8.

Attention is now directed to FIG. 8. With reference to FIG. 8, a computer-implemented method 800 is illustrated that describes one embodiment for managing operating condition parameters in medical devices. The method 800 is performed by at least a processor of a computer system that accesses and interacts with memories and/or data storage devices. For example, the processor at least accesses and reads/writes data to the memory and processes network communications to perform the actions of FIG. 8.

With reference to FIG. 8, at 801, the method 800 is initiated when the end user (usually the wearer of the hood 14 and gown 4) interacts with a remote computer 965 such as a mobile communication device that is configured with a mobile app (iOS and Android) that is “paired with” or otherwise interacts with the medical device (e.g., the hood 14 and/or the ventilation system 100 prior to the medical procedure). In one embodiment, the medical device can be selected by the end user either by selecting from a list of medical devices or scanning the barcode on the medical device before the surgery.

In another embodiment, the end user can either download the end user's fan speed preferences through the remote computer 965 to the processor 902 where the preferences are stored in the data storage 906. In another embodiment, the user can also download and store threshold values for the operating conditions such as, but not limited to, carbon dioxide (CO₂), temperature, humidity, oxygen (O₂), VOC, and/or air pressure levels.

With reference to FIG. 8, at 802, the method 702 is initiated when operating condition parameter sensor 602 and/or 602a monitor the operating conditions within hood 14 (FIG. 7) while the wearer is donning the hood 14. As discussed above, the operating condition parameter sensor 602 and/or 602a can be used to monitor operating parameter conditions such as, but not limited to, carbon dioxide (CO₂), temperature, humidity, oxygen (O₂), VOCs, and/or air pressure levels.

In block 804, in one embodiment, if at least one operating condition parameter has exceeded an operating parameter threshold (e.g., the CO₂ level within the hood 14 has exceeded 1,200 ppm or 2,500 ppm depending upon the operating conditions), the operating condition parameter sensor 602 and/or 602a will determine this high level of CO₂ within hood 14 and forward this information to processor 902.

In block 806, upon determining that at least one operating condition parameter has exceeded its operating condition threshold, the processor 902 in conjunction with printed circuit board (PCB) module 500 can then interact with fan motor 260 in order to adjust the speed of the fan motor 260.

In one embodiment, the fan motor 260 can be adjusted to operate the impeller 454 at a “constant” setting. For example, the wearer may set an initial fan speed at 2 out of 10. If the CO₂ operating parameter condition within hood 14 is detected above 1,200 ppm (or 2.500 ppm depending upon the operating conditions), the processor 902 can also be configured to send a signal to an alarm (not shown) such as an audible alarm (such as a beep) to the wearer and/or a haptic (vibrate) alarm (such as a vibration from the fan motor 260) to provide an alert to the user to inform the user that at least one of the thresholds of an operating condition (carbon dioxide (CO₂), temperature, humidity, oxygen (O₂), VOCs, and/or air pressure levels) has been exceeded. As discussed above, the fan motor 260 can be adjusted to operate the impeller 454 at a desired speed and/or a desired time in order to attempt to correct the operating condition so that the operating parameter condition does not exceed its operating parameter threshold. The processor 902 in conjunction with printed circuit board (PCB) module 500 will then instruct the fan motor 260 to slowly increase the fan speed to 3, 4, or 5 (which is measured by the tachometer 263) until the CO₂ level as measured by the operating condition parameter sensor 602 and/or 602a goes below the operating condition parameter threshold. Finally, the fan speed will stay at this higher level unless manually turned down by the wearer.

In another embodiment, assume that the wearer has set the operating condition parameter for CO₂ to be at 1,200 ppm and the correction or mitigation method is set to “purge”. The wearer also sets the fan speed to 2 out of 10. If the CO₂ operating condition within hood 14 is detected above 1,200 ppm, the ventilation system 100 may be equipped to provide an alarm (such as a beep) to the wearer and the fan motor 260 may provide an alarm (such as a vibration), as discussed above. The processor 1502 in conjunction with printed circuit board (PCB) module 500 will then instruct the fan motor 260 to quickly increase the fan speed to 9 or 10 (which is measured by the tachometer 263) for a short period until the CO₂ level as measured by the operating condition parameter sensor 602 and/or 602a goes below the operating condition parameter threshold. Finally, the fan speed will return to the initially selected level.

In block 808, the operating condition parameter sensor 602 and/or 602a can be used to monitor the operating parameter conditions within the hood 14 to determine if the operating parameter condition has been corrected so that the operating parameter condition is now below its operating condition parameter threshold.

### Computing Device Embodiment

FIG. 9 illustrates an example computing device that is configured and/or programmed as a special purpose computing device with one or more of the example systems and methods described herein, and/or equivalents. The example computing device may be a computer 900 that includes at least one hardware processor 902, a memory 904, and input/output ports 910 operably connected by a bus 908. In one example, the computer 900 may include logic 930 similar to logic/system 800 shown in FIG. 8.

In different examples, the logic 930 may be implemented in hardware, a non-transitory computer-readable medium 937 with stored instructions, firmware, and/or combinations thereof. While the logic 930 is illustrated as a hardware component attached to the bus 908, it is to be appreciated that in other embodiments, the logic 930 could be implemented in the processor 902, stored in memory 904, or stored in disk 906.

In one embodiment, logic 930 or the computer is a means (e.g., structure: hardware, non-transitory computer-readable medium, firmware) for performing the actions described. In some embodiments, the computing device may be a server operating in a cloud computing system, a server configured in a Software as a Service (SaaS) architecture, a smart phone, laptop, tablet computing device, and so on.

The means may be implemented, for example, as an ASIC programmed to predict a product demand. The means may also be implemented as stored computer executable instructions that are presented to computer 900 as data 916 that are temporarily stored in memory 904 and then executed by processor 902.

Logic 930 may also provide means (e.g., hardware, non-transitory computer-readable medium that stores executable instructions, firmware) for storing and measuring operating condition parameters.

Generally describing an example configuration of the computer 900, the processor 902 may be a variety of various processors including dual microprocessor and other multi-processor architectures. A memory 904 may include volatile memory and/or non-volatile memory. Non-volatile memory may include, for example, ROM, PROM, and so on. Volatile memory may include, for example, RAM, SRAM, DRAM, and so on.

A storage disk 906 may be operably connected to the computer 900 via, for example, an input/output (I/O) interface (e.g., card, device) 918 and an input/output port 910 that are controlled by at least an input/output (I/O) controller 940. The disk 906 may be, for example, a magnetic disk drive, a solid-state disk drive, a floppy disk drive, a tape drive, a Zip drive, a flash memory card, a memory stick, and so on. Furthermore, the disk 906 may be a CD-ROM drive, a CD-R drive, a CD-RW drive, a DVD ROM, and so on. The memory 904 can store a process 914 and/or a data 916, for example. The disk 906 and/or the memory 904 can store an operating system that controls and allocates resources of the computer 900.

The computer 900 may interact with, control, and/or be controlled by input/output (I/O) devices via the input/output (I/O) controller 940, the I/O interfaces 918, and the input/output ports 910. Input/output devices may include, for example, one or more displays 970, printers 972 (such as inkjet, laser, or 3D printers), audio output devices 974 (such as speakers or headphones), text input devices 980 (such as keyboards), cursor control devices 982 for pointing and selection inputs (such as mice, trackballs, touch screens, joysticks, pointing sticks, electronic styluses, electronic pen tablets), audio input devices 984 (such as microphones or external audio players), video input devices 986 (such as video and still cameras, or external video players), image scanners 988, video cards (not shown), disks 906, network devices 920, and so on. The input/output ports 910 may include, for example, serial ports, parallel ports, and USB ports.

The computer 900 can operate in a network environment and thus may be connected to the network devices 920 via the I/O interfaces 918, and/or the 1/O ports 910. Through the network devices 920, the computer 900 may interact with a network 960. Through the network, the computer 900 may be logically connected to remote computers 965. Networks with which the computer 900 may interact include, but are not limited to, a LAN, a WAN, and other networks.

### Definitions and Other Embodiments

In another embodiment, the described methods and/or their equivalents may be implemented with computer executable instructions. Thus, in one embodiment, a non-transitory computer readable/storage medium is configured with stored computer executable instructions of an algorithm/executable application that when executed by a machine(s) cause the machine(s) (and/or associated components) to perform the method. Example machines include but are not limited to a processor, a computer, a server operating in a cloud computing system, a server configured in a Software as a Service (SaaS) architecture, a smart phone, and so on). In one embodiment, a computing device is implemented with one or more executable algorithms that are configured to perform any of the disclosed methods.

In one or more embodiments, the disclosed methods or their equivalents are performed by either: computer hardware configured to perform the method: or computer instructions embodied in a module stored in a non-transitory computer-readable medium where the instructions are configured as an executable algorithm configured to perform the method when executed by at least a processor of a computing device.

While for purposes of simplicity of explanation, the illustrated methodologies in the figures are shown and described as a series of blocks of an algorithm, it is to be appreciated that the methodologies are not limited by the order of the blocks. Some blocks can occur in different orders and/or concurrently with other blocks from that shown and described. Moreover, less than all the illustrated blocks may be used to implement an example methodology. Blocks may be combined or separated into multiple actions/components. Furthermore, additional and/or alternative methodologies can employ additional actions that are not illustrated in blocks. The methods described herein are limited to statutory subject matter under 35 U.S.C § 101.

The following includes definitions of selected terms employed herein. The definitions include various examples and/or forms of components that fall within the scope of a term and that may be used for implementation. The examples are not intended to be limiting. Both singular and plural forms of terms may be within the definitions.

References to “one embodiment”, “an embodiment”, “one example”, “an example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

A “data structure”, as used herein, is an organization of data in a computing system that is stored in a memory, a storage device, or other computerized system. A data structure may be any one of, for example, a data field, a data file, a data array, a data record, a database, a data table, a graph, a tree, a linked list, and so on. A data structure may be formed from and contain many other data structures (e.g., a database includes many data records). Other examples of data structures are possible as well, in accordance with other embodiments.

“Computer-readable medium” or “computer storage medium”, as used herein, refers to a non-transitory medium that stores instructions and/or data configured to perform one or more of the disclosed functions when executed. Data may function as instructions in some embodiments. A computer-readable medium may take forms, including, but not limited to, non-volatile media, and volatile media. Non-volatile media may include, for example, optical disks, magnetic disks, and so on. Volatile media may include, for example, semiconductor memories, dynamic memory, and so on. Common forms of a computer-readable medium may include, but are not limited to, a floppy disk, a flexible disk, a hard disk, a magnetic tape, other magnetic medium, an application specific integrated circuit (ASIC), a programmable logic device, a compact disk (CD), other optical medium, a random access memory (RAM), a read only memory (ROM), a memory chip or card, a memory stick, solid state storage device (SSD), flash drive, and other media from which a computer, a processor or other electronic device can function with. Each type of media, if selected for implementation in one embodiment, may include stored instructions of an algorithm configured to perform one or more of the disclosed and/or claimed functions. Computer-readable media described herein are limited to statutory subject matter under 35 U.S.C § 101.

“Logic”, as used herein, represents a component that is implemented with computer or electrical hardware, a non-transitory medium with stored instructions of an executable application or program module, and/or combinations of these to perform any of the functions or actions as disclosed herein, and/or to cause a function or action from another logic, method, and/or system to be performed as disclosed herein. Equivalent logic may include firmware, a microprocessor programmed with an algorithm, a discrete logic (e.g., ASIC), at least one circuit, an analog circuit, a digital circuit, a programmed logic device, a memory device containing instructions of an algorithm, and so on, any of which may be configured to perform one or more of the disclosed functions. In one embodiment, logic may include one or more gates, combinations of gates, or other circuit components configured to perform one or more of the disclosed functions. Where multiple logics are described, it may be possible to incorporate the multiple logics into one logic. Similarly, where a single logic is described, it may be possible to distribute that single logic between multiple logics. In one embodiment, one or more of these logics are corresponding structure associated with performing the disclosed and/or claimed functions. Choice of which type of logic to implement may be based on desired system conditions or specifications. For example, if greater speed is a consideration, then hardware would be selected to implement functions. If a lower cost is a consideration, then stored instructions/executable application would be selected to implement the functions. Logic is limited to statutory subject matter under 35 U.S.C. § 101.

An “operable connection”, or a connection by which entities are “operably connected”, is one in which signals, physical communications, and/or logical communications may be sent and/or received. An operable connection may include a physical interface, an electrical interface, and/or a data interface. An operable connection may include differing combinations of interfaces and/or connections sufficient to allow operable control. For example, two entities can be operably connected to communicate signals to each other directly or through one or more intermediate entities (e.g., processor, operating system, logic, non-transitory computer-readable medium). Logical and/or physical communication channels can be used to create an operable connection.

“User”, as used herein, includes but is not limited to one or more persons, computers or other devices, or combinations of these.

While the disclosed embodiments have been illustrated and described in considerable detail, it is not the intention to restrict or in any way limit the scope of the appended claims to such detail. It is, of course, not possible to describe every conceivable combination of components or methodologies for purposes of describing the various aspects of the subject matter. Therefore, the disclosure is not limited to the specific details or the illustrative examples shown and described. Thus, this disclosure is intended to embrace alterations, modifications, and variations that fall within the scope of the appended claims, which satisfy the statutory subject matter requirements of 35 U.S.C. § 101.

To the extent that the term “includes” or “including” is employed in the detailed description or the claims, it is intended to be inclusive in a manner similar to the term “comprising” as that term is interpreted when employed as a transitional word in a claim.

To the extent that the term “or” is used in the detailed description or claims (e.g., A or B) it is intended to mean “A or B or both”. When the applicants intend to indicate “only A or B but not both” then the phrase “only A or B but not both” will be used. Thus, use of the term “or” herein is the inclusive, and not the exclusive use.

Therefore, provided herein is a new and improved system and method for managing medical devices and medical device consumables, which according to various embodiments of the present invention, offers the following advantages: ease of use; the ability to keep track of operating condition parameters in medical devices; the ability to automatically correct the operating condition parameter in the medical device without user intervention, if needed; the ability to provide feedback regarding the operating condition parameters in the medical device for wearer edification, preventative maintenance, and or product improvement; and the ability to provide audible and haptic (vibrate) alerts.

In fact, in many of the preferred embodiments, these advantages of ease of use, the ability to keep track of operating condition parameters in medical devices, the ability to automatically correct the operating condition parameter in the medical device without user intervention, if needed, the ability to provide feedback regarding the operating condition parameters in the medical device for wearer edification, preventative maintenance, and or product improvement, and the ability to provide audible and haptic (vibrate) alerts are optimized to an extent that is considerably higher than heretofore achieved in prior, known systems and methods for managing operating condition parameters in medical devices.

What is claimed is:

1. A helmetless support and ventilation system for use with surgical hoods and gowns, comprising:
a surgical gown;
a surgical hood operatively connected to the surgical gown, wherein the hood is located over a head and neck area of a wearer such that the head and neck area of the wearer are substantially enclosed within the hood;
a ventilation system located within the surgical gown and the surgical hood for providing ventilation air within the surgical gown and the surgical hood, wherein the ventilation system is retained by shoulders of the wearer of the ventilation system in order to provide ventilation air within the surgical gown and surgical hood; and
an operating parameter measurement assembly operatively connected to the ventilation system, wherein the operating parameter measurement assembly is located within the surgical hood.

2. The helmetless support and ventilation system for use with surgical hoods and gowns, according to claim 1, wherein the ventilation system is further comprised of:
a power module; and
an air flow generation module located adjacent to the power module.

3. The helmetless support and ventilation system for use with surgical hoods and gowns, according to claim 2, wherein the power module is further comprised of:
a battery;
a battery sensor operatively connected to the battery;
a power module identification system operatively connected to the battery:
a fan motor operatively connected to the battery; and
a motor sensor/wireless identification system operatively connected to the fan motor, wherein the motor sensor/wireless identification system includes a tachometer.

4. The helmetless support and ventilation system for use with surgical hoods and gowns, according to claim 3, wherein the air flow generation module is further comprised of:
a fan motor operatively connected to the battery; and
an impeller operatively connected to the fan motor.

5. The helmetless support and ventilation system for use with surgical hoods and gowns, according to claim 4, wherein the operating parameter measurement assembly is further comprised of:
an operating condition parameter sensor; and
a microphone assembly located within the surgical hood.

6. The helmetless support and ventilation system for use with surgical hoods and gowns, according to claim 5, wherein the operating condition parameter sensor is further comprised of:
a carbon dioxide (CO₂) sensor;
a temperature sensor;
a humidity sensor;
an oxygen (O₂) sensor;
a volatile organic compounds (VOCs) sensor; and
an air pressure sensor.

7. The helmetless support and ventilation system for use with surgical hoods and gowns, according to claim 5, wherein the helmetless support and ventilation system is further comprised of:
a processor; and
a printed circuit board (PCB) module operatively connected to the processor, wherein the PBC module is operatively connected to the operating condition parameter sensor such that the processor and the printed circuit board (PCB) module utilize information from the operating condition parameter sensor to determine if a level of an operating condition within the surgical hood has exceeded a threshold level, and
wherein the PBC module is operatively connected to the tachometer such that the processor and the printed circuit board (PCB) module utilize information from the tachometer to determine a speed at which the fan motor is currently operating and compare the current speed to a desired speed at which the fan motor should be operating in order to reduce the operating condition level within the surgical hood to below the threshold level.

8. A ventilation system for use with surgical hoods and gowns, wherein the ventilation system is further comprised of:
a ventilation assembly, wherein the ventilation assembly is retained by shoulders of a wearer of the ventilation system in order to provide ventilation air within a surgical gown and a surgical hood being worn by the wearer; and
an operating parameter measurement assembly operatively connected to the ventilation assembly, wherein the operating parameter measurement assembly is located within the surgical hood.

9. The ventilation system for use with surgical hoods and gowns, according to claim 8, wherein the ventilation assembly is further comprised of:
a power module; and
an air flow generation module located adjacent to the power module.

10. The ventilation system for use with surgical hoods and gowns, according to claim 9, wherein the power module is further comprised of:
a battery;
a battery sensor operatively connected to the battery;
a power module identification system operatively connected to the battery:
a fan motor operatively connected to the battery; and
a motor sensor/wireless identification system operatively connected to the fan motor, wherein the motor sensor/wireless identification system includes a tachometer.

11. The ventilation system for use with surgical hoods and gowns, according to claim 10, wherein the air flow generation module is further comprised of:
a fan motor operatively connected to the battery; and
an impeller operatively connected to the fan motor.

12. The ventilation system for use with surgical hoods and gowns, according to claim 11, wherein the operating parameter measurement assembly is further comprised of:
an operating condition parameter sensor; and
a microphone assembly located within the surgical hood.

13. The ventilation system for use with surgical hoods and gowns, according to claim 12, wherein the operating condition parameter sensor is further comprised of:
a carbon dioxide (CO₂) sensor;
a temperature sensor;
a humidity sensor;
an oxygen (O₂) sensor;
a volatile organic compounds (VOCs) sensor; and
an air pressure sensor.

14. The ventilation system for use with surgical hoods and gowns, according to claim 12, wherein the ventilation system is further comprised of:
a processor; and
a printed circuit board (PCB) module operatively connected to the processor,
wherein the PBC module is operatively connected to the operating condition parameter sensor such that the processor and the printed circuit board (PCB) module utilize information from the operating condition parameter sensor to determine if a level of an operating condition within the surgical hood has exceeded a threshold level, and
wherein the PBC module is operatively connected to the tachometer such that the processor and the printed circuit board (PCB) module utilize information from the tachometer to determine a speed at which the fan motor is currently operating and compare the current speed to a desired speed at which the fan motor should be operating in order to reduce the operating condition level within the surgical hood to below the threshold level.

15. A method of using a helmetless support and ventilation system with surgical hoods and gowns, comprising:
providing a surgical gown;
providing a surgical hood operatively connected to the surgical gown, wherein the hood is located over a head and neck area of a wearer such that the head and neck area of the wearer are substantially enclosed within the hood;
providing a ventilation system located within the surgical gown and the surgical hood for providing ventilation air within the surgical gown and the surgical hood, wherein the ventilation system is retained by shoulders of the wearer of the ventilation system in order to provide ventilation air within the surgical gown and surgical hood; and
providing an operating parameter measurement assembly operatively connected to the ventilation system,
wherein the operating parameter measurement assembly is located within the surgical hood, and
wherein the ventilation system and the operating parameter measurement assembly are utilized to control an operating condition within the surgical hood.

16. The method, according to claim 15, wherein the step of providing a ventilation system is further comprised of the steps of:
providing a power module; and
providing an air flow generation module located adjacent to the power module.

17. The method, according to claim 16, wherein the step of providing a power module is further comprised of the steps:
providing a battery;
providing a battery sensor operatively connected to the battery:
providing a power module identification system operatively connected to the battery;
providing a fan motor operatively connected to the battery; and
providing a motor sensor/wireless identification system operatively connected to the fan motor, wherein the motor sensor/wireless identification system includes a tachometer.

18. The method, according to claim 17, wherein the step of providing an air flow generation module is further comprised of the steps of:
providing a fan motor operatively connected to the battery; and
providing an impeller operatively connected to the fan motor.

19. The method, according to claim 18, wherein the step of providing an operating parameter measurement assembly is further comprised of the steps of:
providing an operating condition parameter sensor; and
providing a microphone assembly located within the surgical hood.

20. The method, according to claim 19, wherein the method is further comprised of the steps of:
providing a processor; and
providing a printed circuit board (PCB) module operatively connected to the processor,
wherein the PBC module is operatively connected to the operating condition parameter sensor such that the processor and the printed circuit board (PCB) module utilize information from the operating condition parameter sensor to determine if a level of an operating condition within the surgical hood has exceeded a threshold level, and
wherein the PBC module is operatively connected to the tachometer sensor such that the processor and the printed circuit board (PCB) module utilize information from the tachometer to determine a speed at which the fan motor is currently operating and compare the current speed to a desired speed at which the fan motor should be operating in order to reduce the operating condition level within the surgical hood to below the threshold level.
